(19) United States
(12) Patent Application Publication
(10) Pub. No.: US 2012/0101476 A1
(43) Pub. Date: Apr. 26, 2012

(54) Transvenous Soaker Catheter Using Microfibers

(76) Inventor: Guy P. Curtis, San Diego, CA (US)

(21) Appl. No.: 13/091,914

(22) Filed: Apr. 21, 2011

Related U.S. Application Data
(63) Continuation-in-part of application No. 12/113,742, filed on May 1, 2008.

(51) Int. Cl. A61M 5/168; A61M 25/10

(52) U.S. Cl. 604/509; 604/96.01; 604/101.05

(57) ABSTRACT

A catheter tip for an infusion catheter has a hollow tubular shaped base member with a pair of annular shaped inflation balloons positioned in parallel on the base member. A plurality of microfibers are mounted on the cylindrical surface section between the balloons. The microfibers are in fluid communication with the lumen of the catheter tip. In operation, the catheter tip is advanced into the vasculature of a patient and to a site in a small vein that is near the target tissue for infusion. The balloons are then inflated to anchor the catheter tip, and fluid medicament is infused through the microfibers. In an alternate embodiment, the microfibers can be replaced with at least one orifice.

This application is a continuation-in-part of application Ser. No. 12/113,742, filed May 1, 2008, which is currently pending. The contents of application Ser. No. 12/113,742 are incorporated herein by reference.

### FIELD OF THE INVENTION

The present invention pertains generally to systems and methods for infusing fluid medicaments into tissue in the vasculature of a patient. More particularly, the present invention pertains to in-dwelling catheters. The present invention is particularly, but not exclusively, useful as a system and its method of use wherein the catheter tip is anchored in a small vessel (e.g. vein) of the vasculature to infuse a fluid medicament from the lumen of the vessel, and through the wall of the vessel, into target tissue surrounding the vessel.

### BACKGROUND OF THE INVENTION

Heretofore, when organs inside the body have required therapeutic treatment, the typical practice has been to orally or intravenously administer medicaments. Typically, these medicaments (i.e. drugs) transit to and from the target organ through the body's blood stream. In many instances, however, this unnecessarily exposes the entire body to the effect. And, it can be quite problematic due to the fact an efficacious concentration of the fluid medicament in one area of the body can be toxic in others.

Not infrequently, it happens that only a portion of an organ's tissue requires therapeutic treatment. Moreover, it may be desirable, and indeed necessary, to treat this portion of organ tissue with relatively high concentrations of a fluid medicament for an extended period of time. An effective way to do this is by infusing a fluid medicament directly into extracellular fluid in the target tissue. Fortunately, with such infusions the effect can remain somewhat localized as the medicament diffuses down its concentration gradient from the site of application to more distant sites in the tissue. Further, the effect may even be limited to only the particular organ and, thus, effectively isolated from the blood stream. Nevertheless, under such circumstances it is necessary that the administration of the fluid medicament be properly controlled.

Various types of catheter systems are commercially available for use in the vasculature of a patient. Typically, each catheter system is specially designed to perform a particular function, or functions, for a specific application. Of particular interest for the present invention are indwelling catheters that can remain in situ in the venous system of a patient's vasculature for extended periods of time.

It is also known that a fluid medicament, when confined to a particular space (volume) within a vessel of the vasculature, will infuse through the vessel wall and into the surrounding tissue. Preferably, the space will be relatively small, and can be established to not adversely affect blood flow through the vasculature. In the event, there are several such locations in the vasculature where a relatively small vein (e.g. left atrial) is adjacent to, or is surrounded by, target tissue that will benefit from the infusion of a fluid medicament.

In light of the above, it is an object of the present invention to provide a transvenous soaker catheter, and its method of use, that is able to selectively deliver substances (i.e. fluid medicaments) at adjustable concentrations into the extracellular fluid, at a particular location in a target organ. Another object of the present invention is to provide a transvenous soaker catheter that effectively limits the exposure of an administered fluid medicament to targeted organ tissue. Still another object of the present invention is to provide a transvenous soaker catheter, and its method of use, that minimizes system toxicity while allowing the local administration of much higher concentrations of a drug (i.e. fluid medicament) in a predetermined area. It is an object of the present invention to provide a device that will create a space within the vasculature of a patient where a fluid medicament can be introduced for subsequent infusion into target tissue surrounding the space. Another object of the present invention is to provide a fluid flow device that will effectively allow a fluid medicament to be infused into target tissue from inside a vessel in the vasculature of a patient. Still another object of the present invention is to provide a catheter tip for an infusion catheter that is easy to use, is relatively simple to manufacture, and is comparatively cost effective.

### SUMMARY OF THE INVENTION

In accordance with the present invention, a system for infusing a fluid medicament into a volume of tissue includes a catheter having a proximal end and a distal end. A source of the fluid medicament is attached in fluid communication with the proximal end of the catheter, and a controller is provided to establish a predetermined fluid flow rate for the flow of the fluid medicament from the source to the catheter. Also, the controller includes a timer that allows control over the time duration of the fluid medicament flow from the source to the catheter.

An infusion member is affixed to the distal end of the catheter. Preferably, the infusion member is an elongated needle like structure that extends in a distal direction from the distal end of the catheter. For one embodiment of the infusion member, it is formed with at least one laser pin hole. In another embodiment, at least one biodegradable fiber extends from the infusion member. For either embodiment, after the fluid medicament has been pumped through the catheter, the fluid medicament leaves the infusion member though the pin hole(s) or the biodegradable fiber(s).

As envisioned for the present invention, the infusion member can be associated with a stabilizing element at the distal end of the catheter. For example, a helical wire (i.e. cork-screw shaped wire) can be attached to the distal end of the catheter to surround the infusion member. As another example, the infusion member itself can be helical shaped.

In the operation of the present invention, the intravenous soaker catheter is maneuvered through the venous system until the infusion member is positioned at an intended target site. The infusion member is then embedded into tissue at the target site, and stabilized. In many instances, the length of the infusion member will be sufficient to stabilize it at the target site. On the other hand, as indicated above, stabilization can also be accomplished by screwing a helical stabilization wire that is mounted on the catheter, into tissue at the target site. The infusion member itself may also be helical shaped and, thus, it can be similarly screwed into tissue at the target site for stabilization.

Once the catheter has been properly positioned, and the infusion member properly stabilized, the controller can be activated. Specifically, in accordance with instructions from the controller, the fluid pump can be operated to infuse fluid medicament from the fluid source into extracellular fluid in the target volume of fluid. This can be done at a predetermined fluid flow rate, for a predetermined time duration. As envisioned for the present invention, the infusion of fluid medicament can be continuously, or periodically, accomplished over extended periods of time (e.g. greater than five minutes).

In accordance with another aspect of the present invention, an infusion catheter includes a tip that can be anchored in the vasculature of a patient. Preferably, the tip is anchored in a small vein of the vasculature (e.g. left atrial), and a fluid medicament is released from the fluid delivery device located at the tip. Once released, the fluid medicament is then infused into target tissue in the proximity of the catheter tip.

Structurally, the catheter tip of the infusion catheter for the present invention has a hollow, tubular shaped base member. Also, there is an annular shaped, inflatable balloon that is mounted on the outer surface of the base member at its distal end. Likewise, an annular shaped, inflatable balloon is mounted on the outer surface of the base member at its proximal end. The two mounted balloons are separated from each other by a distance “d” to establish a cylindrical surface section between them. With this configuration, the proximal end of the base member (i.e. catheter tip) is affixed to the distal end of the infusion catheter, to place their respective lumens in fluid communication with each other.

For a preferred embodiment of the fluid delivery device for the present invention, a plurality of microfibers are mounted on the cylindrical surface section of the base member (catheter tip) for fluid communication with its inner lumen. For this embodiment (i.e. microfibers), the distal end of the base member may either be closed or patent, as desired. In an alternate embodiment of the fluid delivery device for the present invention, microfibers need not be used. Instead, an orifice can be formed at the distal end of the base member through which the fluid medicament can enter the lumen of the vessel where the base member is anchored.

Extracorporeal components for use with the catheter system of the present invention include a source of the fluid medicament that is to be infused into the patient. Also included is an infusion pump for pumping the fluid medicament from the source, through the infusion catheter, and out of the fluid delivery device at the catheter tip. Further, such a system may include a controller that is electronically connected to the infusion pump for establishing a fluid flow rate for the fluid medicament through the system. Additionally, a selector can be incorporated with the controller to selectively change the fluid flow rate between a steady chronic flow rate and a pulsed flow rate.

In an operation of the present invention, the catheter tip is advanced through the vasculature to a site near the target tissue. The balloons are then inflated to anchor the catheter at the site. Typically, the target tissue will be near a vessel, such as a small vein (e.g. left atrial), and the inflated balloons will urge against the walls of this vessel to anchor the catheter tip at the site. Once the catheter tip has been anchored in the vasculature, the infusion pump can be activated to infuse the fluid medicament into the target tissue.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a system for an indwelling, transvenous soaker catheter in accordance with the present invention;

FIG. 2 is a cross section view of tissue adjacent a vein in the venous system with a soaker catheter of the present invention operationally embedded into the tissue;

FIG. 3 is an alternate embodiment of the infusion member for a soaker catheter in accordance with the present invention;

FIG. 4 is a view of an infusion member with a helical screw for stabilizing the infusion member in the venous system;

FIG. 5 is another alternate embodiment showing the infusion member formed as a helical screw;

FIG. 6 is a schematic of an alternate system for an indwelling, transvenous soaker catheter;

FIG. 7 is a schematic view of the catheter tip of the alternate system shown positioned in the vasculature of a patient; and

FIG. 8 is a cross-section view of the catheter tip of the alternate system as seen along the line 8-8 in FIG. 7.

### DESCRIPTION OF THE PREFERRED EMBODIMENTS

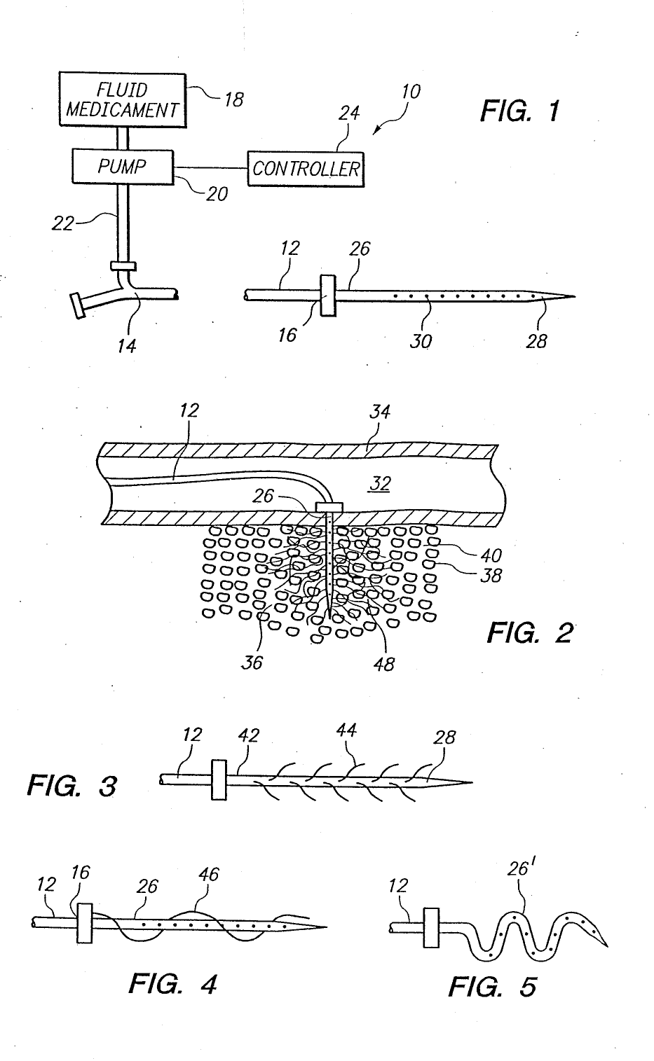

Referring initially to FIG. 1, a system for a transvenous soaker catheter in accordance with the present invention is shown and is generally designated 10. As shown, the system 10 includes an indwelling catheter 12 that has a proximal end 14 and a distal end 16. The system 10 also includes a source 18 of a fluid medicament (i.e. drug), and a pump 20 that can be used to pump the fluid medicament from the source 18 through a fluid line 22. In order to control the flow of this fluid medicament, the system 10 further includes a controller 24 that is electronically connected to the pump 20. In this combination, the controller 24 can be preprogrammed to operate the pump 20 according to a predetermined schedule. Specifically, this schedule can include the fluid flow rate of the fluid medicament being pumped by the pump 20, as well as the time duration of the fluid flow.

Still referring to FIG. 1, it will be seen that the fluid line 22 is connected in fluid communication with the proximal end 14 of the catheter 12. Thus, fluid medicament from the source 18 can be pumped directly to the catheter 12 in accordance with preprogrammed instructions from the controller 24. FIG. 1 also shows that the system 10 includes an infusion member 26 that is affixed to the distal end 16 of the catheter 12. For the purposes of the present invention, the infusion member 26 is preferably an elongated hollow needle 28 that is formed with a plurality of holes 30. As shown, the holes 30 are preferably aligned along the axis of the hollow needle 28. For its use in the system 10, the hollow needle 28 can be made of stainless steel and the holes 30 can be created using well known laser techniques.

Turning now to FIG. 2, the infusion member 26 is shown in its intended operational environment. Specifically, FIG. 2 shows the catheter 12 positioned in the lumen 32 of a vein 34. In this instance, the infusion member 26 of the system 10 has been embedded into the tissue 36 of an organ in the human body (not shown). Further, the tissue 36 in FIG. 2 is shown to anatomically comprise a plurality of cells 38 that are surrounded by extracellular fluid 40. As will be appreciated by the skilled artisan, although the discussion here considers the tissue 36 to be organ tissue, it is to be appreciated that other types of tissue (e.g. muscular tissue) may also be the intended target tissue 36 for therapeutic intervention with the system 10.

An alternative to the infusion member 26 (shown in FIGS. 1 and 2) is an infusion member 42 as shown in FIG. 3. Like the infusion member 26, infusion member 42 incorporates an elongated hollow needle 28. Instead of holes 30, however, the infusion member 42 incorporates biodegradable fibers 44 that are positioned along the length of the needle 28. For use in the system 10, the biodegradable fibers 44 need to be permeable to the fluid medicament that is to be used and, thus, they are in fluid communication with the source 18.

Additional alternate embodiments of components for use with the system 10 are shown in FIGS. 4 and 5. Specifically, FIG. 4 shows a helical shaped wire 46 that is affixed to the distal end 16 of the catheter 12. As intended for the system 10, this helical shaped wire 46 can be screwed into the target tissue 36 to provide additional stabilization for the infusion member 26 during operation of the system 10. Another alternative is shown in FIG. 5. There, an infusion member 26′ is shown configured with a substantially helical shape. In this configuration, the infusion member 26′ can, itself, be screwed into the target tissue 36 for additional stabilization.

In the operation of the system 10 of the present invention, a fluid medicament is selected for use and provided as the fluid source 18. Controller 24 is then programmed for the controlled operation of the pump 20. Specifically, a fluid flow regimen is established for the pump 20, with a prescribed fluid flow rate, and scheduled operational time durations (periodic or continuous). Then, with an infusion member 26 affixed to its distal end 16, the catheter 12 is advanced through the venous system of a patient until the infusion member 26 has been positioned adjacent target tissue 36 at the selected site. The infusion member 26 is then embedded into the target tissue 36 (see FIG. 2), by methods well known in the pertinent art. Lastly, the controller 24 is activated.

As indicated in FIG. 2, operation of the system 10 causes an infused medicament 48 to mix with extracellular fluid 40 in the target tissue 36. Importantly, the concentration of fluid medicament in source 18, as well as the rate, and the time duration of fluid flow provided by pump 20 are controllable. Consequently, the concentration gradient of infused medicament 48 in the target tissue 36 is also controllable.

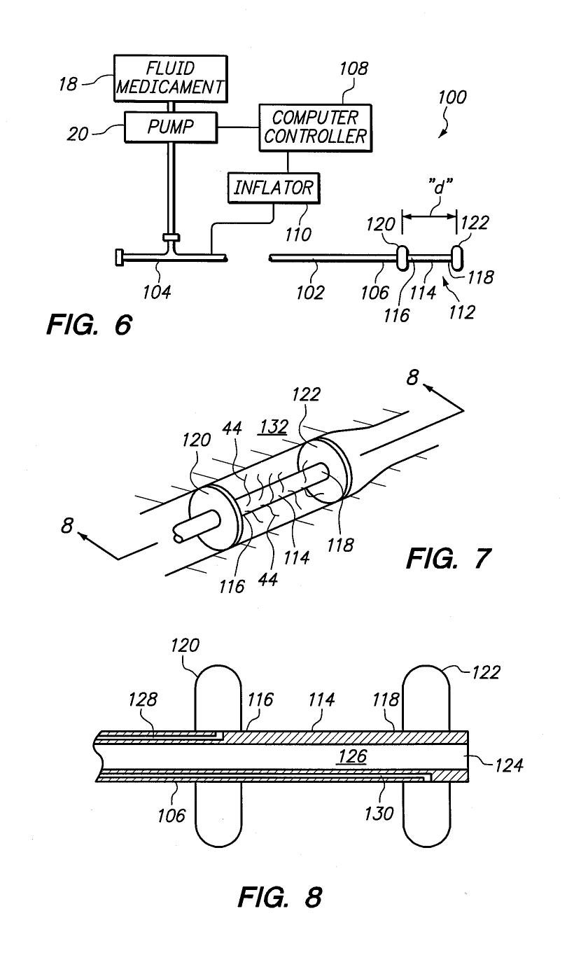

Referring now to FIG. 6 an alternate system for an indwelling, transvenous soaker catheter is shown and is generally designated 100. In detail, the system 100 includes an infusion catheter 102 having a proximal end 104 and a distal end 106. Further, the system 100 includes a source of fluid medicament 18 that is connected via a pump 20 to the proximal end 104 of the system 100. As shown, a computer/controller 108 is connected with the pump 20 for selectively operating the pump 20. Also, the computer/controller 108 is connected with an inflator 110 for selectively operating the inflator 110. Like the pump 20, the inflator 110 is connected in fluid communication with the proximal end 104 of the infusion catheter 102.

At the distal end 106 of the infusion catheter 102, FIG. 6 shows there is a catheter tip that is generally designated 112. More specifically, the catheter tip 112 includes a tubular-shaped body member 114 that has a proximal end 116 and a distal end 118. FIG. 6 also indicates that the proximal end 116 of the body member 114 is connected in fluid communication with the distal end 106 of the infusion catheter 102, and that an inflatable balloon 120 is mounted on the proximal end 116 of the catheter tip 112. Also, FIG. 6 shows that an inflatable balloon 122 is mounted on the distal end 118 of the catheter tip 112 and aligned substantially parallel to the inflatable balloon 120. As shown for the system 100, the proximal inflatable balloon 120 is located on the catheter tip 112 at a distance “d” from the distal inflatable balloon 122.

FIG. 7 shows that, for a preferred embodiment of the system 100, a plurality of microfibers 44 can be mounted on the body member 114, between the inflatable balloons 120 and 122. Also, FIG. 8 shows an alternate embodiment for the system 100 wherein an infusion port 124 is formed at the distal end 118 of the catheter tip 112. For these embodiments, respectively, the microfibers 44 (FIG. 7) and the infusion port 124 (FIG. 8) are each in fluid communication with the lumen 126 that runs the length of the infusion catheter 102 and the catheter tip 112. It is to be further appreciated that in another embodiment of the system 100, the infusion port 124 may be closed. If so, the embodiment of the system 100 that incorporates the microfibers 44 (shown in FIG. 7) will be used. Still referring to FIG. 8, it will be seen that an inflation lumen 128 is provided for the proximal inflatable balloon 120, and that an inflation lumen 130 is provided for the distal inflatable balloon 122. Both inflation lumen 128 and inflation lumen 130 are connected in fluid communication with the inflator 110. Accordingly, inflation balloon 120 and inflation balloon 122 can be selectively and individually inflated by the computer/controller 108 as desired by the operator.

In an operation of the system 100, the catheter tip 112 is advanced through the vasculature of a patient (not shown) until it has been advanced as far as possible. An example of a typical route for the advancement of the catheter tip 112 in the vasculature begins in the subclavian vein, with a subsequent advancement through the right atrium, the coronary sinus, and into the left atrial vein. This, however, is only exemplary, as the system 100 may be used in any organ, and the catheter tip 112 can be wedged into a suitably sized vein at any desired location. In the event, once the catheter tip 112 has been positioned at a site 132 in the vasculature (e.g. see FIG. 7), the inflatable balloons 120 and 122 can be inflated in accordance with instructions from the computer/controller 108. Preferably, both of the balloons 120 and 122 are inflated in order to assure a firm anchorage for the catheter tip 112 and to prevent fluid backwash. The present invention, however, also envisions the possible inflation of only one of the balloons 120 or 122.

Once the catheter tip 112 has been positioned as desired in the vasculature of a patient, the computer/controller 108 is activated to operate infusion pump 20 for an infusion of medicament from the source 18. As envisioned for the present invention, this infusion of fluid medicament will be made through either the microfibers 44, or through the infusion port 124, or simultaneously through both. Further, in accordance with pre-programmed instructions, the infusion of medicament may be either at a steady chronic rate or it can be pulsed with intermittently high fluid flow rates.

While the particular Transvenous Soaker Catheter Using Microfibers as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A catheter system for infusing a fluid medicament into target tissue in the vasculature of a patient which comprises:
an infusion catheter having a distal end and a proximal end, wherein the infusion catheter is formed with a lumen extending between the distal and proximal ends of the catheter;
a catheter tip affixed to the distal end of the infusion catheter, wherein the catheter tip includes a fluid transfer device for directing the fluid medicament from the lumen of the catheter to a surface of the target tissue, for infusion of the fluid medicament through the surface and into the target tissue; and
an inflatable balloon mounted on the catheter tip, wherein the balloon is selectively inflatable for engagement thereof with a vessel wall in the vasculature, to anchor the catheter in the vasculature during an infusion of the fluid medicament.

2. A system as recited in claim 1 wherein the catheter tip is tubular shaped and has a proximal end and a distal end, with the proximal end of the catheter tip being affixed to the distal end of the catheter, and wherein the inflatable balloon is located at the distal end of the catheter tip.

3. A system as recited in claim 2 wherein the inflatable balloon is a first balloon and the system further comprises a second inflatable balloon mounted on the catheter tip at a distance “d” proximal to the first inflatable balloon to establish a cylindrical surface section therebetween.

4. A system as recited in claim 3 further comprising a plurality of microfibers mounted on the cylindrical surface section of the catheter tip in fluid communication with the lumen of the infusion catheter for use as the fluid transfer device.

5. A system as recited in claim 1 wherein the fluid transfer device is an orifice formed at the distal end of the catheter tip.

6. A system as recited in claim 1 further comprising:
a source of the fluid medicament; and
an infusion pump connected in fluid communication between the fluid medicament source and the proximal end of the infusion catheter for pumping fluid medicament from the source and into the lumen of the catheter.

7. A system as recited in claim 6 further comprising a controller electronically connected to the infusion pump for establishing a fluid flow rate for the fluid medicament from the source and into the lumen of the catheter.

8. A system as recited in claim 7 further comprising a selector incorporated into the controller for selectively changing the fluid flow rate between a steady chronic flow rate and a pulsed flow rate.

9. A system as recited in claim 1 further comprising an inflator connected in fluid communication with the inflatable balloon for selective inflation of the balloon.

10. A catheter tip apparatus for use with an infusion catheter to infuse a fluid medicament into target tissue in the vasculature of a patient which comprises:
a hollow, tubular shaped base member having a proximal end, a distal end, and an outer surface extending therebetween, wherein the base member is formed with an inner lumen extending between the proximal and distal ends thereof for receiving the fluid medicament therein;
a first inflatable balloon, wherein the first inflatable balloon is annular shaped and is mounted on the outer surface of the base member at the distal end of the base member;
a second inflatable balloon, wherein the second inflatable balloon is annular shaped and is mounted on the outer surface of the base member at the proximal end of the base member and at a distance “d” from the first inflatable balloon to establish a cylindrical surface section therebetween; and
a means for selectively inflating the first and second balloons to anchor the catheter tip in the vasculature during an infusion of the fluid medicament into the target tissue.

11. An apparatus as recited in claim 10, wherein the infusion catheter has a distal end and a proximal end with a lumen extending therebetween, and wherein the proximal end of the catheter tip is affixed to the distal end of the infusion catheter for fluid communication between the lumen of the infusion catheter and the lumen of the catheter tip, and wherein the apparatus further comprises:
a source of the fluid medicament; and
an infusion pump connected in fluid communication between the fluid medicament source and the proximal end of the infusion catheter for pumping fluid medicament from the source and through the lumen of the catheter to the catheter tip.

12. An apparatus as recited in claim 11 wherein the catheter tip includes a fluid transfer device.

13. An apparatus as recited in claim 12 wherein the fluid transfer device comprises a plurality of microfibers mounted on the cylindrical surface section of the catheter tip in fluid communication with the lumen of the catheter tip.

14. An apparatus as recited in claim 12 wherein the fluid transfer device is an orifice formed at the distal end of the catheter tip.

15. An apparatus as recited in claim 11 further comprising a controller electronically connected to the infusion pump for establishing a fluid flow rate for the fluid medicament from the source and into the lumen of the catheter.

16. An apparatus as recited in claim 15 further comprising a selector incorporated into the controller for selectively changing the fluid flow rate between a steady chronic flow rate and a pulsed flow rate.

17. An apparatus as recited in claim 16 further comprising an inflator connected in fluid communication with the first and second inflatable balloons for selective inflation thereof.

18. A method for infusing a fluid medicament into target tissue in the vasculature of a patient which comprises the steps of:
advancing a catheter tip to a site near the target tissue in the vasculature, wherein the catheter tip is affixed to a distal end of an infusion catheter, and wherein the catheter tip includes a fluid transfer device and is formed as a hollow, tubular shaped base member, with a first inflatable balloon mounted at a distal end of the base member and a second inflatable balloon mounted at a proximal end of the base member in parallel with the first inflatable balloon, with a distance “d” therebetween;
inflating the first and second balloons to anchor the catheter tip in the vasculature during an infusion of the fluid medicament; and
pumping the fluid medicament from a source and through the infusion catheter to the catheter tip, for directing the fluid medicament from the fluid transfer device of the catheter tip to a surface of the target tissue, for infusion of the fluid medicament through the surface and into the target tissue.

19. A method as recited in claim 18 further comprising the step of establishing a fluid flow rate for the fluid medicament from the source and through the infusion catheter.

20. A method as recited in claim 19 further comprising the step of selectively changing the fluid flow rate between a steady, chronic flow rate and a pulsed flow rate.
